(19) United States
(12) Patent Application Publication
(10) Pub. No.: US 2012/0316509 A1
(43) Pub. Date: Dec. 13, 2012

(54) DISCHARGE DEVICE HAVING A LOCKING ELEMENT

(75) Inventors: Ralph Egon Kayser, Luzern (CH); William Brem, Muri (CH)

(73) Assignee: MEDMIX SYSTEMS AG, Rotkreuz (CH)

(21) Appl. No.: 13/577,526

(22) PCT Filed: Nov. 25, 2010
(86) PCT No.: PCT/CH2010/000300

§ 371 Date: Aug. 7, 2012

(30) Foreign Application Priority Data
Feb. 12, 2010 (CH) 172/10

(51) Int. Cl. A61M 5/00

(52) U.S. Cl. 604/210

(57) ABSTRACT

The invention relates to a discharge device for discharging a fluid product, for example, in the form of a syringe. The discharge device comprises a housing (1), which bounds a reservoir (16), and a piston (23), which is slidably arranged in the housing (1) in a longitudinal direction in order to discharge the fluid product distally from the reservoir (16) through an outlet opening (13). A window opening (15) is formed in a circumferential side wall (11) of the housing (1). An insert element (3) is inserted into said window opening (15) perpendicularly to the longitudinal direction. The insert element (3) protrudes into the reservoir (16). In this way, a motion of the piston (23) can be limited in the proximal direction in order to prevent the piston (23) from falling out, and the metering of the fluid product can be made easier in that the insert element (3) establishes a detachable locking connection with an advancing element (27) for the piston (23).

### TECHNICAL FIELD

The present invention relates to a discharge device for discharging a fluid product, with a housing and a piston slidable therein.

### PRIOR ART

Discharge devices in the form of syringes have been generally known for a long time. A commercially available syringe comprises a syringe body with a distal outlet opening and a piston unit that from a proximal end can be slid into the syringe body. The piston unit comprises a piston rod with a piston arranged distally on it, which piston is sealingly slidable in the interior of the syringe body. In order to receive a fluid in the syringe the piston unit can be withdrawn in the proximal direction, as a result of which, in the syringe body, negative pressure arises and the fluid is sucked in through the distal outlet opening. During subsequent sliding of the piston unit in the distal direction the fluid is discharged through the outlet opening.

The above is associated with a danger of the piston unit unintentionally being withdrawn too far from the syringe body, and in this process falling from said syringe body so that fluid received in the syringe is spilled, thus contaminating the surroundings. Furthermore, frequently it is desirable to discharge only a certain quantity or dose of the fluid from the syringe. With the use of commercially available syringes such dosing or portioning is possible only inaccurately by reading a printed-on scale. There is always the danger of too much fluid being unintentionally dispensed. Conversely, when taking up the fluid there is a problem in that, because of the negative pressure present in the syringe body, the piston during release springs back a little in the direction of the outlet opening, and consequently the finally drawn-up quantity of fluid is smaller than desired.

CA 2 036 940, WO 2009/144085, U.S. Pat. No. 2,856,925, and U.S. Pat. No. 4,874,385 each describe a syringe in which a stop element is affixed in a region of the piston rod, which region is situated outside the syringe housing. When the piston unit is advanced, the stop element comes to rest against the proximal housing end, thus preventing further advance. By a corresponding arrangement of the stop element on the piston rod the dose to be administered can be set. However, movement of the piston unit in the proximal direction is not limited with these syringes.

U.S. Pat. No. 2,373,520, U.S. Pat. No. 2,875,761 and U.S. Pat. No. 2,474,496 disclose syringes on whose proximal housing ends on the outside a spring element is arranged. This spring element is designed, outside the syringe housing, to snap into locking notches on the piston rod, and in this manner to indicate the completed discharge of a determined dose. US 2009/0308891 discloses various embodiments of a double syringe with a double piston. In one embodiment the proximal end of the double syringe comprises locking catches that engage locking notches of the two piston rods of the double piston. In another embodiment a separate locking rod is connected to the double piston, which locking rod extends parallel to the piston rods and comprises locking notches. Again, the proximal end of the double syringe comprises locking catches that engage these locking notches. However, accidental removal of the piston unit from the housing is also possible with all of the above-mentioned syringes.

U.S. Pat. No. 2,707,954 discloses a further syringe of this type with a spring element in which syringe, however, complete removal of the piston unit from the housing is prevented. However, the design of this syringe overall is elaborate and complex. Furthermore, the syringe is awkward to handle.

U.S. Pat. No. 4,386,606 and U.S. Pat. No. 4,711,637 disclose syringes in which the piston unit is blockable on the housing by means of a clamping element. Furthermore, the clamping element forms a proximal end stop for the piston, thus preventing full withdrawal of the piston from the housing. A proximal piston end stop that is used to prevent removal of a piston unit from a housing is also disclosed in U.S. Pat. No. 772,114. The proximal end stop elements shown in these documents are all arranged on the outside, at the proximal end of the housing, so as to be detachable. This results in the danger of the end stop element shifting during handling with the discharge device, or even becoming undone.

WO 2009/125353 describes a discharge device with a reservoir and a piston affixed to a piston rod. In a sidewall that extends around the piston and the piston rod there is a window opening into which a locking element has been inserted. This locking element engages the jagged piston rod and is used to slide the piston in the distal direction.

In the application device shown in DE 39 25 681 a jagged piston rod is slidable in longitudinal direction by means of a spring element that can be operated by the user through a lateral opening.

### PRESENTATION OF THE INVENTION

It is an object of the present invention to provide a discharge device that is designed in such a manner that it avoids completely removing the piston from the housing, and/or allows the user to achieve simple portioning of the discharged product. Furthermore, the discharge device is to make possible safe and simple handling and a design that is as simple as possible.

This object is met by a device with the features of claim 1. Further embodiments are provided in the dependent claims.

The present invention thus provides a discharge device for discharging a fluid product, comprising

a housing, which delimits a reservoir for the fluid product, with a circumferential sidewall, an open proximal housing end, and a distal housing end with an outlet opening, and

a piston which is slidably arranged in the housing along a longitudinal direction in order to discharge the fluid product from the reservoir through the outlet opening,

wherein a window opening is formed in the side wall of the housing, and

wherein the discharge device comprises an insert element that is inserted into the window opening transversely to the longitudinal direction, thus protruding into the reservoir.

Depending on its concrete design, the insert element makes it possible to prevent complete removal of the piston from the housing, and/or to perceptibly indicate to the user the discharge of a determined quantity of the fluid.

Below, indications of directions are used as follows. The longitudinal direction designates the direction of sliding of the piston. In each case, the distal direction is the direction in which the piston is advanced towards an outlet opening for the purpose of discharging a fluid received in the device. The proximal direction designates the direction that is opposite to the aforesaid.

The discharge device can, in particular, be designed as a syringe with a piston that is sealingly slidable in the housing. The housing can comprise a classical cylindrical syringe shape, or it can, for example, also form a cartridge whose outer housing shape differs from the classical syringe shape. The piston can directly delimit the reservoir for the fluid, or it can indirectly act on the reservoir, wherein the reservoir is, for example, delimited by a bag or a bellows-like container accommodated in the housing. In such a case it is not mandatory for the piston to rest sealingly against the sidewall of the housing, even though this is the case in preferred embodiments. The distal housing end can, for example, be formed by a wall that extends perpendicularly or obliquely to the longitudinal direction, or it can have some other shape. The outlet opening preferably points in the distal direction along the longitudinal direction, but it can also extend at an angle to the longitudinal direction.

The term “fluid” refers to any flowing state. The term “fluid” thus includes low-viscosity fluids, viscous fluids such as oils or gels, emulsions, suspensions or powders. The fluid to be discharged can, in particular, be a medicament, a component of a medicament to be mixed, or a medical or non-medical adhesive component.

The window opening is preferably arranged adjacent to the proximal housing end, in particular closer to the proximal housing end than to the distal housing end. Said window opening is preferably circumferentially rimmed by the sidewall and, in particular, in the proximal direction is preferably completely delimited by the sidewall. However, the window opening can also be open in the proximal direction, as long as in this direction as a result of its shape it forms an end stop for the insert element, which end stop affixes the insert element in the proximal direction. Advantageously, the insert element is arranged as an entity in a region of the sidewall, which region is spaced apart from the proximal housing end; in other words it is fully distally-spaced-apart from the proximal housing end so that the insert element does not proximally protrude beyond the proximal housing end. Preferably, the insert element is inserted into the window opening so as to be perpendicular to the longitudinal direction. Preferably, towards the outside, said insert element finishes so as to be flush with the housing in order to prevent unintended removal and make possible simple handling of the device, as well as providing an aesthetically pleasing appearance.

Preferably, the reservoir delimited by the housing is, at least in some regions, in the form of a circular cylinder, wherein the cylinder axis defines the longitudinal direction. The insert element is then advantageously inserted from the outside into the window opening in a radial direction perpendicular to the aforesaid. The window opening then extends in circumferential direction of the circular cylinder over an angular range of less than 180°, preferably less than 90°, in particular of 30-60°.

In order to ensure that the insert element is held in the window opening, said insert element can comprise at least one engaging element. In this arrangement the engaging element can be used to snap the insert element into the window opening. Advantageously, in this arrangement the engaging element engages an inside of the sidewall so as to snap into place. Furthermore, the insert element can be designed so that after first snapping into place into the window opening it can no longer be removed from said window opening. In this way, for example for reasons associated with hygiene, it is possible to guarantee single use of the discharge device. However, in other embodiments, the insert element can also be detachably inserted into the window opening.

In a preferred embodiment the insert element is designed so that it forms an end stop for the piston during movement of the piston in the direction of the proximal housing end. This prevents unintentional removal of the piston from the housing.

Preferably, the piston is connected to an advancing element that at the proximal housing end protrudes from the housing, e.g. in the form of a piston rod. In this arrangement the piston and the advancing element can be detachable from each other or can be permanently connected to each other and, in particular, formed in a single piece. The advancing element preferably comprises at least one first locking structure, and the insert element comprises a second locking structure.

The locking structures are designed in at least one position of the advancing element along the longitudinal direction to enter a detachable locking connection in order to render difficult any further sliding of the advancing element in longitudinal direction relative to the housing. In this manner, reaching this position during advancement of the advancing element is perceptibly indicated to the user so that the user recognises that a determined portion of the fluid has been discharged. Conversely, when the user withdraws the advancing element, in this way a particular quantity of the fluid drawn into the discharge device can be indicated to the user. Furthermore, in this manner it is possible, during withdrawal of the advancing element in the proximal direction and subsequent release, to prevent said advancing element from moving some distance in the distal direction as a result of negative-pressure build-up.

Usually, the first locking structure and the second locking structure are movable relative to each other in one direction of engagement in order to jointly establish the locking connection. This direction of engagement preferably extends both transversely to the longitudinal direction and transversely to the direction in which the insert element has been inserted into the window opening. In this manner, during engagement and disengagement of the locking connection, forces act in such a manner that they cannot press the insert element from the window opening.

Advantageously, the first locking structure and the second locking structure are designed such that the locking connection can be detached with the application of an axial detachment force along the longitudinal direction. Particularly preferably, the first locking structure and the second locking structure are furthermore designed in such a manner that this detachment force can be directed both in the proximal direction and in the distal direction. Consequently, the advancing element can be further advanced in the distal direction in order to, by overcoming an increased pressure resistance, discharge a further portion, or by overcoming an increased tension force the advancing element can be further withdrawn in order to take up a further portion. In this arrangement, the axial detachment force in the distal direction can differ from the axial detachment force in the proximal direction.

The advancing element can, in particular, comprise a plurality of first locking structures that are arranged so as to be spaced apart in axial direction. The first locking structures then enter a detachable locking connection with the second locking structure in different positions of the advancing element in that during axial movement of the advancing element the first locking structures relative to the housing successively snap into the second locking structure of the insert element. Consequently, the user can discharge the fluid in several well-defined portions from the discharge device. In this arrangement, the first locking structures can, in particular, be arranged at regular spacing on the advancing element.

At least two of the first locking structures can advantageously be designed differently relative to each other such that the axial detachment force acting along the longitudinal direction, which detachment force is required for detaching the locking connection of the respective first locking structure from the second locking structure, is different in each case.

The locking structures can be designed so that for at least one locking connection they still permit certain movement, or play, of the advancing element along the longitudinal direction over a determined axial movement region, without the locking connection being disconnected as a result of this. In this case it is preferable for this movement range in the region of the distal end stop position of the piston to be larger than in other regions. In other words, the piston is slidable to a distal end stop position in which the piston in the distal direction comes to rest against the housing so that further movement of the piston and of the advancing element relative to the housing in the distal direction is prevented. A selected first locking structure is then designed in such a manner that it enters a locking connection with the second locking structure if the piston is located in the region of its distal end stop position. The axial movement region of the advancing element for this locking connection is then greater than it is for at least one other locking connection of one of the remaining first locking structures to the second locking structure. For this purpose the corresponding first locking structure can, in particular, be designed to be longer in relation to the longitudinal direction than at least one other of the first locking structures. This makes it possible to press the piston to the distal end stop even after the locking connection has snapped into place.

In a concrete embodiment the first locking structure can comprise at least one sloping surface that is inclined relative to the longitudinal direction, which sloping surface is designed to interact with the second locking structure. Preferably, the first locking structure comprises two contrarily inclined sloping surfaces which during movement of the advancing element in each case interact in opposing directions with the second locking structure. The inclinations of these sloping surfaces can differ, in order to achieve different axial detachment forces in the distal and proximal directions.

In particular, the first locking structure can comprise a locking notch in the advancing element, and the second locking structure then comprises at least one locking catch that in the locking position engages the locking notch. Preferably, the first locking structure comprises two locking notches arranged on opposite sides of the advancing element, and the second locking structure correspondingly comprises two locking catches that engage the locking notches from opposite sides.

In particular, the second locking structure can comprise two flexibly designed arms that are arranged transversely to the longitudinal direction so as to be opposite each other and in each case comprise a locking catch pointing towards the other arm. The first locking structure is then designed in such a manner that these two locking catches engage the first locking structure from two opposite sides, in order to form the locking connection.

In concrete embodiments the advancing element can comprise a region that comprises a cross-shaped cross section with four webs that are arranged perpendicularly to each other and that extend in longitudinal direction. The first locking structure is then preferably formed on at least one of these webs.

In a preferred embodiment the insert element comprises a main section on which, along the longitudinal direction and spaced apart from each other, three pairs of arms are formed that protrude into the reservoir, which arms are arranged in pairs transversely to the longitudinal direction opposite each other, wherein the arms of the pairs arranged distally and proximally in axial directions in each case comprise an engaging element that extends transversely to the axial direction from the insert element towards the outside, and wherein the arms of the pair arranged in between in each case comprise a locking catch that extends transversely to the axial direction and towards the inside relative to the insert element.

The device can, in particular, also be designed as a multiple applicator with several reservoirs, e.g. as a double syringe or multiple syringe. Each of these reservoirs then comprises a piston that is connected to an advancing element. The advancing elements are then connected by their proximal ends. In this case it is sufficient, if only the sidewall of one of the reservoirs comprises a window opening with an insert element of the type described above.

### BRIEF DESCRIPTION OF THE DRAWINGS

Preferred embodiments of the invention are described below with reference to the drawings that are only intended for explanation and are not to be interpreted as being limiting. The following are shown in the drawings:

FIG. 1 a perspective exploded view of a discharge device according to the invention according to a first embodiment;

FIG. 2 a perspective view of the discharge device shown in FIG. 1 in its assembled state;

FIG. 3 a first lateral view of the discharge device shown in FIG. 1;

FIG. 4 a second lateral view of the discharge device shown in FIG. 1, from a direction of view that is perpendicular when compared to that of FIG. 3;

FIG. 5 a section view in the plane V-V of the discharge device shown in FIG. 3;

FIG. 6 a partial section view in the plane VI-VI of the discharge device shown in FIG. 5, with the piston withdrawn from the housing up to a proximal end stop;

FIG. 7 a partial section view in the plane VI-VI of the discharge device shown in FIG. 5, with the piston advanced into the housing up to a distal end stop;

FIG. 8 a first lateral view of a discharge device according to the invention according to a second embodiment;

FIG. 9 a second lateral view of the discharge device shown in FIG. 8 from a direction of view that is perpendicular when compared to that of FIG. 8;

FIG. 10 a lateral view of a discharge device according to the invention according to a third embodiment; and

FIG. 11 a perspective exploded view of a discharge device according to the invention according to a fourth embodiment.

### DESCRIPTION OF PREFERRED EMBODIMENTS

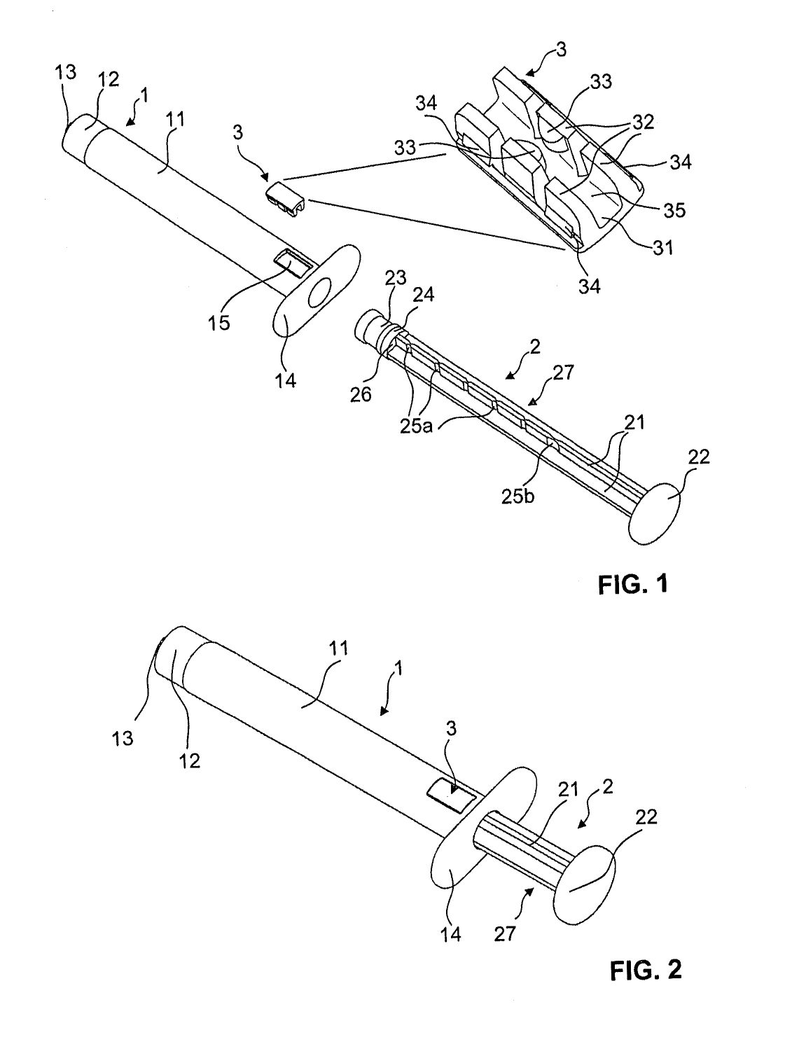

FIGS. 1 to 7 show a discharge device according to the invention according to a preferred embodiment. The discharge device of this embodiment is a syringe which is, for example, used for administering a medicament or for discharging an adhesive. The discharge device comprises a housing 1 in which a piston unit 2 is slidably arranged along an axial direction.

The housing 1, hereinafter also referred to as the “syringe body”, is largely defined by a circumferential sidewall 11 that forms a hollow cylinder, which sidewall 11 comprises a proximal end and a distal end. In this arrangement, the sidewall 11 delimits a cylindrical reservoir 16 that extends in its interior. As a result of its cylindrical form, in this arrangement the reservoir 16 defines a longitudinal direction (axial direction) and a direction of the discharge device, which direction extends radially to the aforesaid, as well as a reservoir centre axis. Following on from the distal end of the sidewall wall 11 an essentially cylindrical head part 12 is arranged that comprises an axial through-opening that ends in a distal outlet opening 13 arranged centrally in the head part. In this arrangement, the outlet opening 13 protrudes in the distal direction beyond the head part 12 towards the outside, wherein said outlet opening 13 can comprise a Luer cone or some other connection structure so that, for example, an injection needle or an accessory part, for example a spray nozzle, can be affixed to the housing 1. In the reservoir 16 a fluid product can be received, which fluid product can, for example, be a medicament, an adhesive or some other fluid.

On the proximal end of the housing 1, on two diametrically opposing sides of the sidewall 11, two holding wings 14 that radially protrude towards the outside are arranged. These holding wings 14 facilitate handling of the discharge device for the user, in that, for example, during advancing of the piston unit 2 into the housing 1 in each case a finger can be placed on the distal side of each holding wing 14 while the piston unit 2 is advanced by means of thumb pressure.

In the region of the proximal housing end within the sidewall 11 on a side perpendicularly facing the holding wings 14 there is a lateral window opening 15 which in this design extends in circumferential direction of the sidewall 11 over an angular range of approximately 45°. The window opening 15 is rectangular in shape, wherein the longer sides of the rectangle in each case extend along the longitudinal direction. In this embodiment, the window opening 15 is fully edged by the sidewall 11. In this arrangement, in particular the reservoir 16 is made accessible, through the window opening 15, from a radial direction.

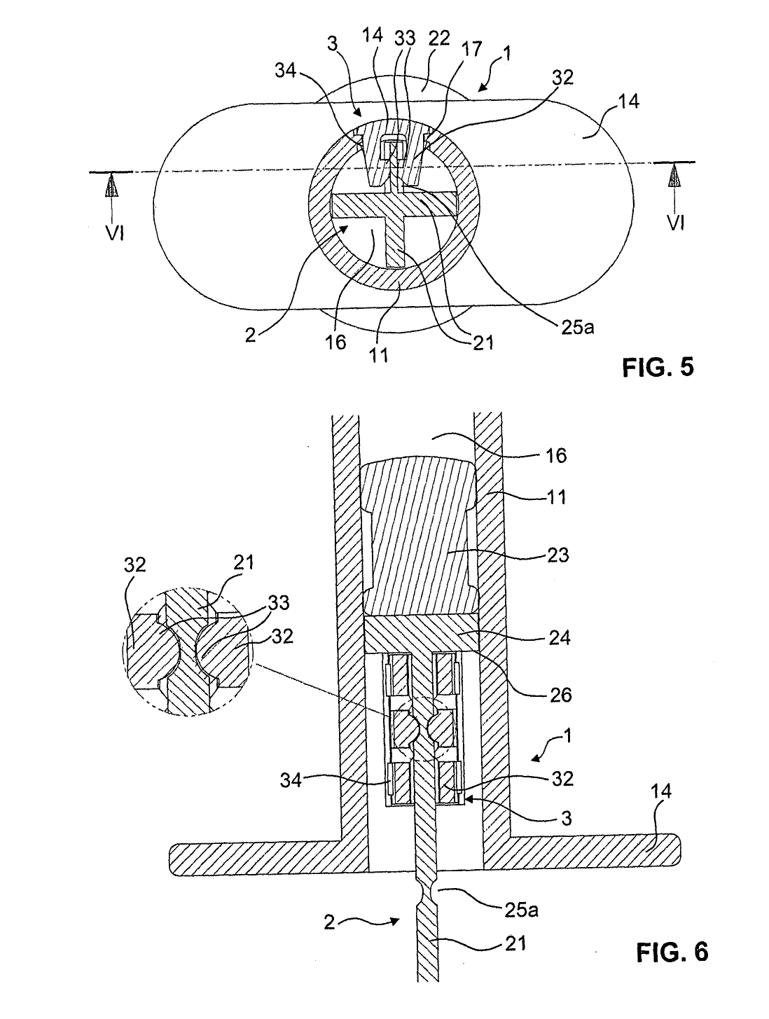

In the edge region of the opening 15 the sidewall 11 can comprise flanges 17 (FIG. 5), which in each case extend along the two longitudinal sides of the rectangular opening 15 in longitudinal direction. In radial direction these flanges 17 are thinner than the sidewall 11 and are arranged approximately in the middle of the sidewall 11 in relation to the radial direction.

The piston unit 2 overall comprises an elongated shape; in this exemplary embodiment, it comprises a distally arranged piston 23, an end plate 24 connected thereto, a piston rod 27 designed in one piece with the end plate 24, which piston rod 27 forms an advancing element for the piston, as well as a proximally arranged pressure plate 22.

In the embodiment shown the piston rod 27 comprises a cross-shaped cross sectional area with four webs 21 that are arranged perpendicularly to each other, that extend along the longitudinal direction, and that can also be referred to as “longitudinal ribs”. The piston rod 27 extends along a large part of the axial length of the piston unit 2.

On the proximal end of the piston rod 27 the planar pressure plate 22 is arranged which extends perpendicularly to the axial direction, which pressure plate 22 has an enlarged outside diameter when compared to that of the remaining piston unit 2. The pressure plate 22 serves, in particular, as a thumb rest for the user when advancing the piston unit.

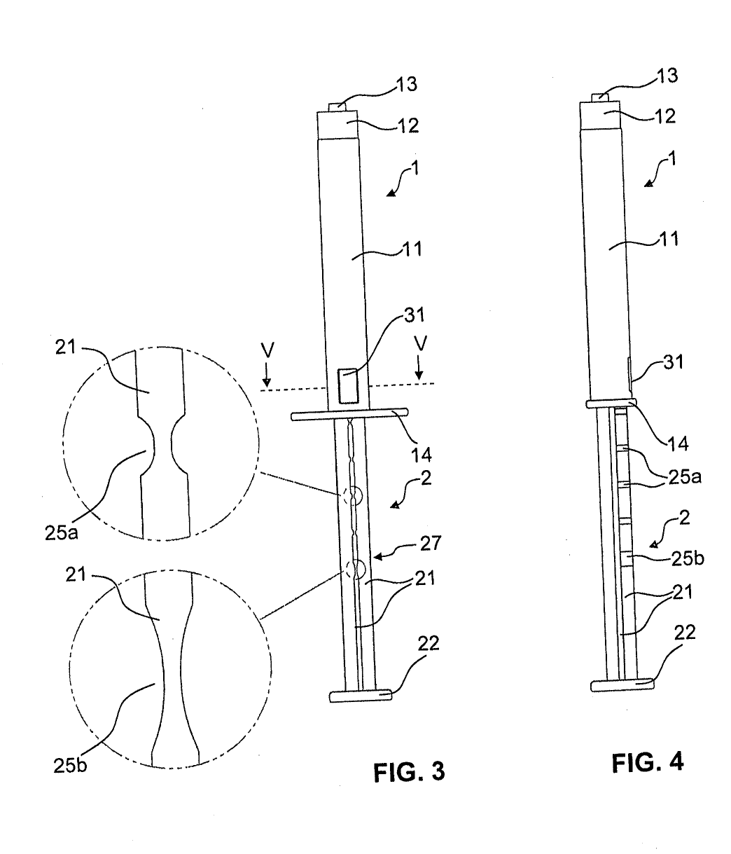

As is, for example, shown in FIGS. 1 and 3, one of the webs 21 in this exemplary embodiment comprises a multitude of locking notches 25a and 25b. The locking notches 25a, 25b are arranged regularly spaced apart along the longitudinal direction. In this arrangement in each case the locking notches 25a, 25b represent a local tapering-off of the web 21, wherein tapering is symmetrical relative to the two opposite sides of the web 21. In this arrangement the locking notches 25a, 25b in each case extend in radial direction, continuously and evenly over the entire longitudinal rib 21, which means that the thickness of the longitudinal rib in radial direction in each position, and in particular also in the region of the locking notches 25a, 25b, does not change. In the top view of the web 21 with locking notches 25a, 25b from a radial direction the locking notches 25a, 25b are in each case of a rounded design, as shown in FIG. 3, thus representing opposite recesses of the web 21 in the form of circular segments or elliptical segments. In particular, the locking notches 25a, 25b in each case comprise a sloping surface that is inclined in the distal direction (and that is curved) as well as an inclined surface that is inclined in the proximal direction. In each case the locking notches 25a or 25b form a locking structure.

On the distal end of the piston unit 2 the piston 23 is arranged. The piston 23 can be permanently connected to the piston rod 27 or it can represent a separate component that can be detached from the remaining piston unit 2. In the present example the piston 23 is connected to an end plate 24. In the distal direction the end plate 24 comprises a pressure area that is designed to slide the piston 23 in the housing 1. In the proximal direction the end plate 24 forms an end stop surface 26 that is connected to the longitudinal ribs 21.

The piston unit 2 is slid from the direction of the proximal housing end into the reservoir 16. In this arrangement, the piston 23 seals the reservoir 16 in the proximal direction so that it is fluid-proof. Consequently, said piston 23 is suitable for pressing through the outlet opening 13 the fluid product received in the reservoir 16, and thus to discharge it from the discharge device. When it is fully advanced into the housing 1, the piston unit 2 protrudes from the housing 1 on the proximal housing end. In this arrangement the webs 21 of the piston rod 27 extend in radial direction almost to the sidewall 11. With the piston unit 2 fully advanced into the housing 1 an end surface of the piston 23, which end surface points in the distal direction, comes to rest against an end stop of the housing 1, which end stop is directed in the proximal direction. Preferably, in such a distal end stop position of the piston 2 relative to the housing 1 the fluid product received in the reservoir 16 is essentially fully discharged from the housing 1. This end stop of the housing 1 can, for example, be formed by a transition region between the sidewall 11 and the head part 12, or by a housing region, which extends in radial direction, adjacent to the outlet opening 13.

In order to prevent the piston unit from being able to be completely pulled from the housing, and in order to facilitate discharging defined portions, an insert element 3 has been inserted into the window opening 15. In the present embodiment the insert element 3 comprises a main section 31, which is essentially rectangular in shape, and is curved towards the outside in accordance with the curvature of the sidewall 11, and finishes so as to be flush with the sidewall. In particular, the main section 31 essentially comprises the form and dimensions of the opening 15 of the housing 1. On the radial inside of the main section 31 three pairs of elastic arms 32 are formed that extend radially inwards into the reservoir 16, wherein the two arms 32 of a pair in each case are arranged transversely to the longitudinal direction and opposite each other in radial direction. The respective arms 32 of a pair define a joint channel 35 of the insert element 3, which channel 35 extends in longitudinal direction.

Overall the insert element 3 is designed to form a positive-locking fit with the housing 1. To this effect the arms 32 arranged on the distal end and on the proximal end of the insert element 3, in each case on their outer side, which in circumferential direction points towards the rim of the opening 15, comprise an engaging element 34. The engaging elements 34 comprise, in particular, a holding surface that is directed radially outwards. These holding surfaces in each case extend parallel to the sidewall 11 in circumferential direction. On their side opposite the holding surface the engaging elements 34 in each case comprise a sloping surface that connects the holding surface to the arm 32. In each case these sloping surfaces extend at an angle to the sidewall 11 and to the radial direction.

When inserting the insert element 3 into the opening 15, the two arms 32, which in each case are arranged so as to be opposite each other, are slightly pushed towards each other until the engaging elements 34 have moved beyond the rim of the opening 15, and the insert element 3 snaps into the opening 15. In this arrangement, the sloping surfaces of the engaging elements 34 facilitate insertion of the insert element into the opening 15 and make it possible for the engaging elements 34 to snap in on the inside of the sidewall 11. The radially-outward pointing holding surfaces of the engaging elements 34 then form an end stop on the inside of the sidewall 11 on the flanges 17, and consequently the insert element 3 is held in the opening 15.

Inserting the insert element 3 into the opening 15 is furthermore facilitated in that the outsides of the arms 32 overall in each case are slightly inclined towards the channel 35. Furthermore, outward-pointing corners of the pairs of arms arranged proximally and distally on the insert element 3 can be rounded, as shown in FIG. 1, in order to further facilitate inserting the insert element 3 into the opening 15.

The flanges 17 of the sidewall 11 make it possible to design the insert element 3 in such a manner that the outside of the main section 31 is flush with the outside of the sidewall 11, when the insert element 3 has been inserted into the opening 15 (FIG. 5). After the insert element 3 has snapped into the window opening 15, consequently any unintended removal of the insert element 3 by the user can be prevented. Furthermore, the flange 17 makes it possible for the engaging elements 34 in the inserted state of the insert element 3 not to protrude into the reservoir 16.

The pair of arms 32 of the insert element 3, which pair is arranged in the middle relative to the longitudinal direction, in the region of the free ends of the arms comprises two locking catches 33 that face each other. In radial direction the locking catches 33 in the state of the insert element 3 inserted into the housing 1 are arranged closer to the reservoir centre axis when compared to the engaging elements 34. Said locking catches 33 comprise a semicircular form that is complementary to the form of the locking notches 25a of the piston unit 2. In this arrangement, the locking catches 33 clearly protrude into the channel 35 of the insert element 3, which channel 35 is formed by the arms 32. The two opposite locking catches 33 together with the arms 32, on which they are formed in each case, form a locking structure.

When the insert element 3 is inserted into the housing 1, the arms 32 protrude in radial direction through the opening 15 into the reservoir 16. By means of this inward protrusion of the arms 32 the insert element 3 provides a proximal end stop for the piston unit 2, which end stop prevents complete removal of the piston unit 2 from the housing 1, as is shown in FIGS. 5 and, in particular, 6. When the piston unit 2 is withdrawn from the housing 1, the end stop surface 26 of the end plate 24 comes to rest against the arms 32 distally arranged on the insert element 3, and consequently the axial movement of the piston unit 2 relative to the housing is effectively limited in the proximal direction. In an alternative embodiment the insert element 3, as a result of the arms 32 protruding into the reservoir 16, could furthermore also form a distal end stop for the piston unit 2, which end stop delimits axial advancing of the piston unit 2 in the distal direction. The piston unit 2 would then in the region of the piston rod 27 comprise a corresponding radially-outward protruding end stop element which would be arranged in a proximal region vis-à-vis the insert element 3.

In the state in which the insert element 3 is inserted into the housing 1, the web 21 of the piston unit 2, which web 21 comprises the locking notches 25a, 25b, extends through the channel 35. Consequently, during sliding of the piston unit 2 within the housing 1 the locking catches 33 of the insert element 3 successively, from the opposite sides of the web 21, snap into place into the locking notches 25a, 25b. In order to jointly enter the locking connection, the locking notches 25a, 25b and the locking catches 33 are movable relative to each other in a direction of engagement that extends transversely to the longitudinal direction and transversely to the direction in which the insert element 3 has been inserted into the window opening 15. To this effect the arms 32, which comprise the locking catches 33, are advantageously designed so as to be slightly flexible. Depending on the movement of the piston unit 2 in the distal or in the proximal direction, in this process the locking catches 33 come to rest against the sloping surfaces of the locking notches 25a, 25b, which sloping surfaces are directed in the proximal direction or in the distal direction. Consequently, advancing or withdrawing the piston unit 2 at these locking positions of the piston unit 2 relative to the housing 1 is perceptibly rendered more difficult to the user. This can, for example, be used to indicate to the user the completed discharging or drawing up of a determined dose. Further advancing or withdrawing of the piston unit 2 when a locking position has been reached is only possible again after a determined detachment force has been overcome.

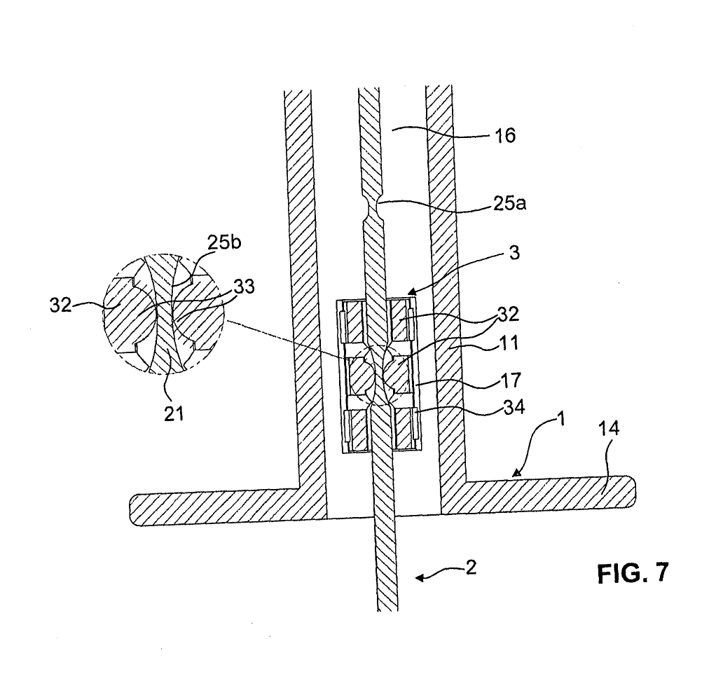

In the snapped-in position of the locking catch 33 in one of the locking notches 25a, 25b, movement of the piston unit 2 relative to the housing 1 (“play”) is possible over a certain axial region. The locking notches 25a are designed in such a manner that this axial movement region is minimal. In contrast to the above, the locking notch 25b arranged closest to the pressure plate 22 comprises a design that is axially extended when compared to the other locking notches 25a (see FIG. 3), and consequently the axial movement range of the locking notch 25b in the locking position is enlarged (FIG. 7). Thus, in a position in which the locking catches 33 snap into the locking notch 25b, a certain play between the piston unit 2 and the housing 1 in axial direction is possible. Preferably, the extended locking notch 25b is arranged on the piston rod 27 in such a manner that it then establishes a locking connection with the locking catch 33, when the piston unit 2 is in a distal end stop position relative to the housing 1. The locking notch 25b arranged and extended in such a manner ensures that the piston unit 2 assumes a locking position in the distal end stop position even with tolerances resulting from the manufacturing process.

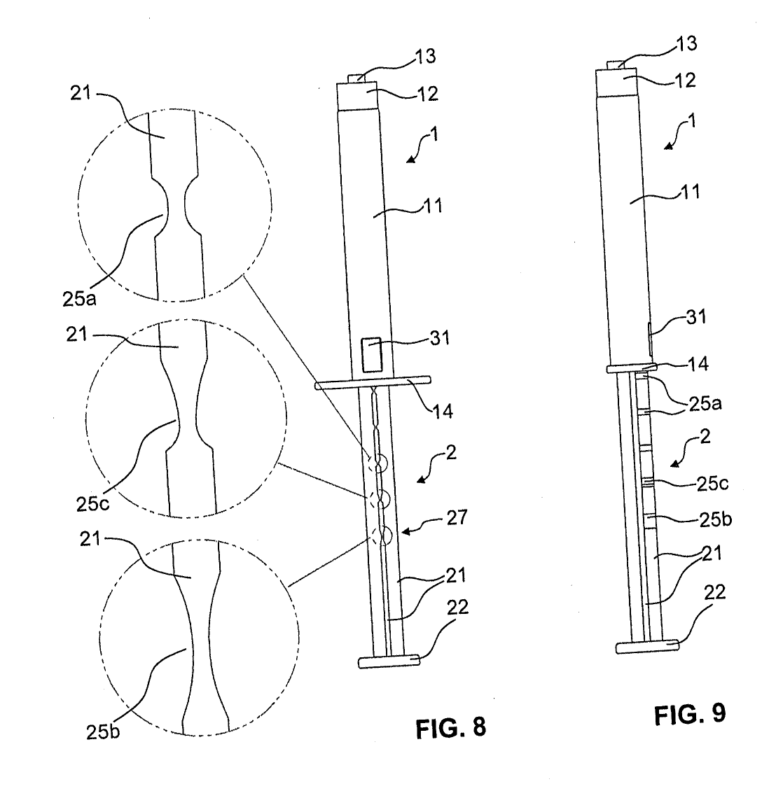

FIGS. 8 and 9 show a second embodiment of the discharge device, in which the locking notch 25c, which in the proximal direction is arranged in comparison to all the locking notches 25a, 25b, 25c so as to be next but one to the pressure plate 22, comprises a special form. The locking notch 25c, like the locking notches 25a and 25b, is also designed so as to be rounded. However, the locking notch 25c is designed so as to be asymmetric relative to the axial direction in that the sloping surface on the distal side of the locking notch 25c extends so as to be significantly more shallow relative to the reservoir centre axis than is the case on the proximal side. When the piston unit 2 is advanced into the housing 1, snapping into place thus takes place perceptibly more slowly and gently. This indicates to the user, for example, that a last remaining dose unit in the discharge device has been reached. During withdrawal of the piston unit 2 from the housing 1, as a result of this shallower distal sloping surface of the locking notch 25c, a reduced detachment force of the locking connection is perceptible by the user.

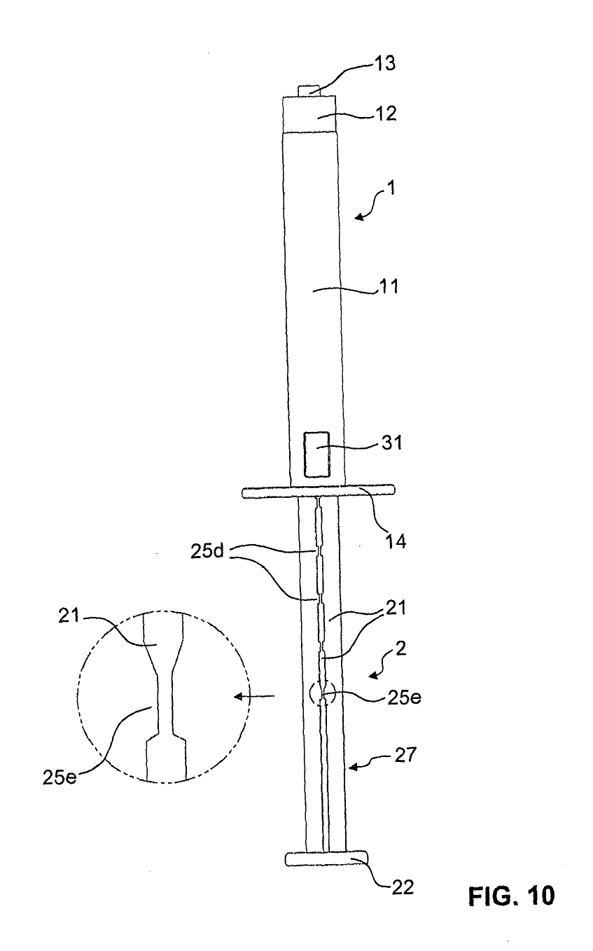

FIG. 10 shows a further embodiment, where the locking notches 25d and 25e are not of a rounded but of an angular design. The angular design of these locking notches 25d, 25e changes the snapping into place, perceived by the user, of the piston 2 in the insert element 3 and the detachment force for detaching the locking connection when compared to the rounded design of the locking notches 25a, 25b. In this embodiment the locking notch 25e arranged rearmost in the proximal direction differs from the other locking notches 25d by its asymmetric design relative to the axial direction. This asymmetric design of the locking notch 25e results in a snap-in behaviour that differs when compared to that of the other locking notches 25d and in this manner indicates to the user completion of sliding of the piston unit 2 or completion of administering the last dose. Furthermore, during advancing of the piston unit 2 the detachment force for detaching this locking connection is reduced.

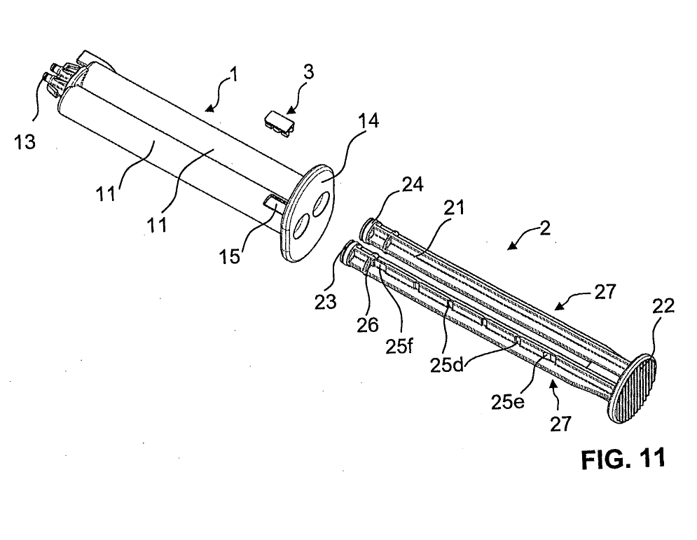

FIG. 11 shows an embodiment in which the discharge device is designed as a double syringe. In this embodiment the housing 1 comprises two parallel reservoirs, each defined by a circumferential sidewall 11, which reservoirs are each used to take up a fluid. Each reservoir leads into an outlet opening 13 to which, for example, a shared mixing element (not shown in the figure) can be affixed, in order to mix the substances of the two reservoirs, which substances are to be discharged. The piston unit 2 is designed as a double piston in which the two elongated piston rods 27 are interconnected by way of the pressure plate 22. In this embodiment, locking notches 25d, 25e and 25f are designed only on one of the two piston rods 27. Correspondingly, only in one of the sidewalls 11 is there an opening 15 into which an insert element 3 can be inserted. The locking notches 25d, 25e and 25f each comprise an angular shape, wherein the locking notches 25f and 25e, which are arranged frontmost and rearmost in axial direction, are each designed so as to be elongated when compared to the other locking notches 25d. Furthermore, the locking notch 25e, which is arranged closest to the pressure plate 22, comprises an asymmetric design.

In the embodiments described the housing 1, the piston unit 2 and the insert element 3 are in each case designed as one piece and are made from a plastic material in an injection moulding process. In this arrangement, in particular, the piston unit 2 can have been produced in a two-component injection moulding process and can comprise a softer piston 23 when compared to the piston rod 27 and to the pressure plate 22. However, the piston unit 2 can also be designed in two parts or several parts, wherein then, in particular, the piston 23 can represent a separate part. Advantageously, the insert element 3 comprises a material that matches the requirements. Thus, for example, it is less brittle than the housing 1.

The invention is, of course, not limited to the above-mentioned exemplary embodiments, and a multitude of variations are possible. For example, the insert element can also be designed so as to differ from the described embodiment, and can, for example, instead of being held by engaging elements that radially hold the insert element in the sidewall 11 from the inside, be held to the housing 1 by means of holding brackets from the outside. It is also possible to provide a cartridge in which the fluid product is held. The cartridge would then have been inserted into the housing and in the interior would comprise a proximal sealing piston, which could be advanced by the piston unit, in order to discharge the fluid. In another embodiment, the fluid could also be held in a compressible bag that has been inserted into the housing. Instead of locking catches, the insert element could, furthermore, also comprise locking notches, and correspondingly, the piston unit, instead of comprising locking notches, could comprise locking catches. A multitude of further modifications are imaginable.

### LIST OF REFERENCE CHARACTERS

1 Housing
11 Sidewall
12 Head part
13 Outlet opening
14 Holding wing
15 Lateral opening
16 Reservoir
17 Flange
2 Piston unit
21 Longitudinal rib
22 Pressure plate
23 Piston
24 End plate
25a Rounded locking notch
25b Rounded extended locking notch
25c Rounded asymmetric locking notch
25d Angular locking notch
25e Angular asymmetric locking notch
25f Angular extended locking notch
26 End stop surface
27 Piston rod
3 Insert element
31 Main section
32 Arm
33 Locking catch
34 Engaging element
35 Channel

1. A discharge device for discharging a fluid product, comprising
a housing, which delimits a reservoir for the fluid product, with a circumferential sidewall, an open proximal housing end, and a distal housing end with an outlet opening,
a piston, which is slidably arranged in the housing along a longitudinal direction in order to discharge the fluid product from the reservoir through the outlet opening, and
an advancing element, connected to the piston, which advancing element at the proximal housing end protrudes from the housing,
wherein a window opening is formed in the sidewall of the housing,
wherein the discharge device comprises an insert element that has been inserted into the window opening transversely to the longitudinal direction, thus protruding into the interior of the housing,
wherein the advancing element comprises at least one first locking structure, and the insert element comprises a second locking structure, and wherein the first and the second locking structures are designed in at least one position of the advancing element to enter a detachable locking connection in order to render difficult any sliding of the advancing element in longitudinal direction relative to the housing, and
wherein the first locking structure and the second locking structure are movable relative to each other in a direction of engagement that extends transversely to the longitudinal direction and transversely to a direction in which the insert element has been inserted into the window opening in order to, together, establish the locking connection.

2. The discharge device according to claim 1, wherein the insert element comprises at least one engaging element in order to snap the insert element into the window opening.

3. The discharge device according to claim 1, wherein the insert element is designed so as to be flush with the housing towards the outside.

4. The discharge device according to claim 1, wherein the insert element forms an end stop for the piston during movement of the piston in the direction of the proximal housing end.

5. The discharge device according to claim 1, wherein the first locking structure and the second locking structure are designed such that the locking connection can be detached with the application of an axial detachment force along the longitudinal direction.

6. The discharge device according to claim 5, wherein the first locking structure and the second locking structure are designed such that the locking connection can be detached both in the proximal and in the distal directions with the application of an axial detachment force along the longitudinal direction, wherein the axial detachment force in the distal direction differs from the axial detachment force in the proximal direction.

7. The discharge device according to claim 1, wherein the advancing element comprises a plurality of first locking structures arranged so as to be spaced apart from each other in longitudinal direction, with said first locking structures being designed in different positions of the advancing element to enter a detachable locking connection with the second locking structure.

8. The discharge device according to claim 7, wherein at least two of the first locking structures are designed differently relative to each other such that an axial detachment force acting along the longitudinal direction, which detachment force is required for detaching the locking connection of the respective first locking structure from the second locking structure, is different in each case.

9. The discharge device according to claim 7,
wherein the first locking structures and the second locking structure (32, 33) are designed so that for at least one locking connection they permit movement of the advancing element relative to the housing over a determined axial movement region without the locking connection being disconnected as a result of this,
wherein the piston is slidable to a distal end stop position in which the piston in the distal direction comes to rest against the housing so that further movement of the piston and of the advancing element relative to the housing in the distal direction is prevented, and
wherein a selected first locking structure is designed in such a manner that it enters a locking connection with the second locking structure when the piston is located in the region of its distal end stop position, and
wherein the axial movement region of the advancing element for this locking connection is greater than it is for at least one other locking connection of one of the remaining first locking structures to the second locking structure.

10. The discharge device according to claim 1, wherein the first locking structure comprises at least one locking notch, and the second locking structure comprises at least one locking catch.

11. The discharge device according to claim 1, wherein the second locking structure comprises two flexibly designed arms that are arranged transversely to the longitudinal direction so as to be opposite each other and in each case comprise a locking catch pointing towards the other arm, and wherein the first locking structure is designed in such a manner that the two locking catches engage the first locking structure from two opposite sides in order to form the locking connection.

12. The discharge device according to claim 1, wherein the insert element comprises a main section on which, spaced apart from each other along the longitudinal direction, three pairs of arms are formed that protrude into the reservoir, wherein the arms of each pair are arranged transversely to the longitudinal direction opposite each other, wherein the arms of the pairs that are arranged distally and proximally in longitudinal direction in each case comprise an engaging element that extends transversely to the longitudinal direction from the insert element towards the outside, and wherein the arms of the pair arranged in between in each case comprise a locking catch that extends transversely to the longitudinal direction and towards the inside relative to the insert element.

13. The discharge device according to claim 1, wherein the housing delimits a first reservoir for a first fluid product, and a second reservoir for a second fluid product,
wherein in the first reservoir a first piston is slidably arranged in order to discharge the first fluid product through a first outlet opening from the first reservoir, and wherein in the second reservoir a second piston is slidably arranged in order to discharge the second fluid product through a first outlet opening from the first reservoir,
wherein the first piston is connected to a first advancing element that on the proximal housing end protrudes from the housing, wherein the second piston is connected to a second advancing element that on the proximal housing end protrudes from the housing, and wherein the first advancing element and the second advancing element are proximally interconnected with each other,
wherein the window opening is formed in the sidewall of the housing towards the first reservoir, and
wherein the insert element is inserted into the window opening transversely to the longitudinal direction, thus protruding into the first reservoir.
